(19) United States
(12) Patent Application Publication
(10) Pub. No.: US 2024/0327451 A1
(43) Pub. Date: Oct. 3, 2024

(54) Synthesis of Delta 9,11 Steroids

(71) Applicant: SANTHERA PHARMACEUTICALS (SCHWEIZ) AG, Pratteln (CH)

(72) Inventors: Gabriel Schäfer, Allschwil (CH); Lauso Olivieri, Ariccia (IT); Marco Martin, Abano Terme (IT)

(21) Appl. No.: 18/682,035

(22) PCT Filed: Jul. 28, 2022
(86) PCT No.: PCT/EP2022/071145

§ 371 Date: Feb. 7, 2024

(30) Foreign Application Priority Data
Aug. 12, 2021 (EP) 21191143.3

(51) Int. Cl. C07J 7/00; A61K 31/573

(52) U.S. Cl. CPC C07J 7/009; A61K 31/573

(57) ABSTRACT

The present invention is directed to a process for the preparation of Δ9,11 steroids by deoxygenation of 9,11-epoxy steroids using HI. The disclosed process selectively forms Δ9,11 steroids such as Vamorolone and can also be used for steroid synthesis of Δ9,11 steroids in pharmaceutical purity.

Described is a process for the preparation of Δ9,11 steroids by deoxygenation of 9,11-epoxy steroids using HI. The disclosed process selectively forms Δ9,11 steroids such as Vamorolone and can also be used for steroid synthesis of Δ9,11 steroids in pharmaceutical purity.

### BACKGROUND OF THE INVENTION

Glucocorticoids are standard of care for many inflammatory conditions, but longterm use is associated with a broad array of side effects. Recently, it was found that Δ9,11 steroid analogs hold promise as a source of safer agents for treating chronic inflammatory disorders.

Typical glucocorticoids like cortisol, prednisone and prednisolone have Δ9,11 steroid counterparts, such as Tirilazad, VBP1 and Anecortave. For such Δ9,11 steroids new syntheses are of need.

Furthermore, the synthesis of corticosteroids having therapeutic utility, such as mometasone, metametasone and beclometasone, requires functionalization of the C-9 and C-11 positions of the steroid molecule. The functionality is generally introduced via Δ9,11 steroid intermediates. The synthesis of Δ9,11 steroids having therapeutic utility, and the synthesis of Δ9,11 steroid intermediates is, thus, of growing importance.

Methods for preparing Δ9,11 steroids, i.e. steroids having a 9,11-double bond, are known in the art. For example, an 11-hydroxy steroid can be converted to the corresponding mesylate (by treating the steroid with mesyl chloride) which is transformed into a Δ9,11 steroid via an elimination reaction. However, the respective prior art methods are not regiospecific in the case of starting from 11-hydroxy steroids and typically lead to mixtures of Δ9,11 steroid containing high and unwanted amounts of Δ11,12 steroids. Separation of these regio-isomeric products is difficult, generally requiring laborious physical separation procedures, resulting in increased costs and lower yields.

Other synthesis strategies are known as well: U.S. Pat. No. 5,399,727 discloses the synthesis of a specific Δ9,11 steroid from a 9-hydroxy steroid with chlorosulfonic acid in an organic solvent. EP 0 969 012 discloses a process for the regioselective dehydration of 11-hydroxy steroids using PCl₅, PCl₃, POCl₃ or either SO₂Cl₂ and imidazole, or PPh₃ and CCl₄. These processes are quite harsh, require toxic chemicals and lead to unintended side-products and impurities.

Vamorolone (17α,21-Dihydroxy-16α-methylpregna-1,4,9(11)-triene-3,20-dione) is an Active Pharmaceutical Ingredient (API), which is a clinical candidate, e.g. for treating DMD. Vamorolone is, thus, a new Δ9,11 steroid of high therapeutic interest.

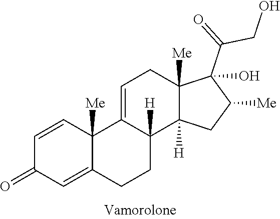

Vamorolone is currently produced from the commercially available 3TR (Tetraene acetate)—see Scheme 2. In step a, TMS imidazole, MeMgCl and THF are added to 3TR, with subsequent addition of CuAC₂, H₂O, DMPU, MeMgCl and THF in step b. Under treatment with peracetic acid in Toluene from compound 2 the intermediate 3 is formed in step c. After treatment with NaHSO₃ and TFA (step d), EtOAc and heptane (step e) and acetonitrile trituration (step f) HBr in CH₂Cl₂ is added (step g) and MeOH (step h) is used for crystallization to form Acetyl-Vamorolone 4. Acetyl-Vamorolone is deacetylated with K₂CO₃ in MeOH, followed by HCl to obtain Vamorolone (step i). The synthesis is disclosed in Bioorganic & Medicinal Chemistry, Volume 21, Issue 8, 15 Apr. 2013, Pages 2241-2249.

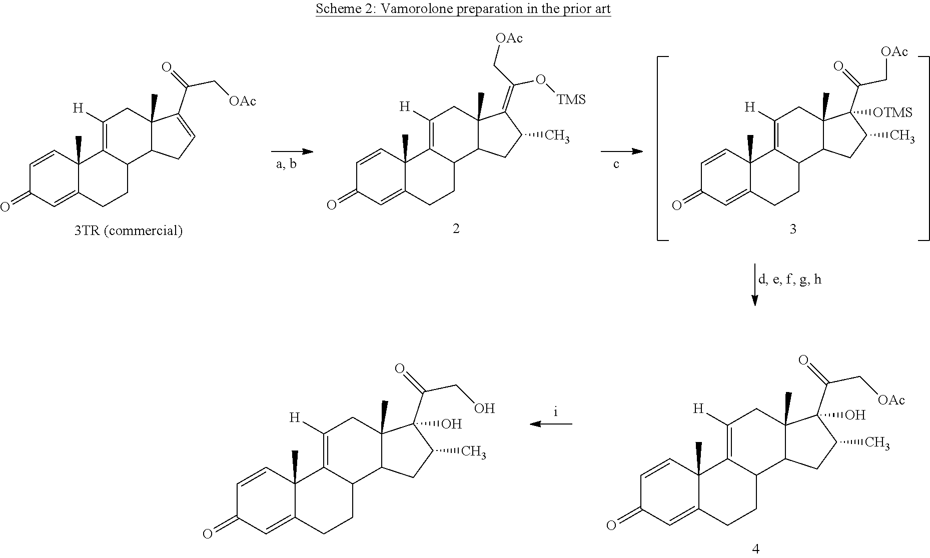

This process is difficult to control, requires the use of several toxic and hazardous chemicals and thus represents an unattractive synthesis of Vamorolone.

Overall, there is a need for a more straight forward and safe way of producing Vamorolone and other Δ9,11 steroids in high yields, and in controlled pharmaceutical purity eliminating the presence of numerous impurities and side-products (e.g. steroid-side-products) in the final product.

### SUMMARY OF THE INVENTION

The present invention fulfils this need by providing a convenient, non-toxic and quantitative synthesis of Vamorolone and other Δ9,11 steroids. The new synthesis is surprisingly simple and significantly reduces steroid-side-products. For the synthesis of Vamorolone and other Δ9,11 steroids HI is used to deoxygenate 9,11 epoxide steroid precursors, such as the protected, e.g. acetylated, commercial 8-DM or the unprotected commercial 8-DM ((1S,2S,13R,14R,15S,17S)-14-hydroxy-14-(2-hydroxyacetyl)-2,13,15-trimethyl-18-oxapenta-cyclo [8.8.0.0^{1.17}.0^{2.7}.0^{11,15}]octadeca-3,6-dien-5-one). Δ9,11 steroids, such as 8-DM, are commercially available and protection groups (to be attached to the 21-OH moiety) can be attached using conventional synthesis means available in the art.

Accordingly, the present invention provides a process for preparing a Δ9,11 steroid of the formula I,

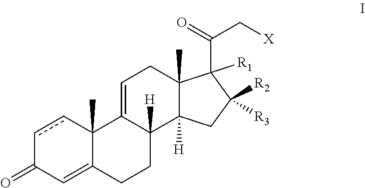

wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine; the tertiary amine throughout the present specification may be part of a linear or cyclic substructure.

Preferred is a process for preparing a Δ9,11 steroid of the formula Ia,

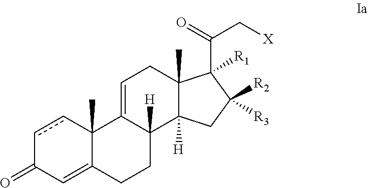

wherein:

The dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₈ alkyl, C₅-C₁₂ cycloalkyl, aryl, substituted aryl, heteroaryl or C₁-C₆ alkoxy, or NR^{II}, wherein NR^{II} is a tertiary amine, wherein the tertiary amine is part of a linear or cyclic substructure.

More preferred is a process for preparing a Δ9,11 steroid of the formula Ib,

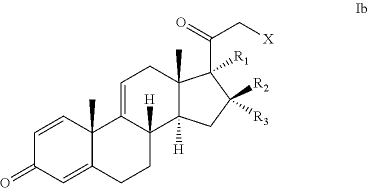

wherein:

R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, substituted aryl, heteroaryl or C₁-C₆ alkoxy, or NR^{II}, wherein NR^{II} is a tertiary amine, wherein the tertiary amine is part of a linear or cyclic substructure.

Even more preferred is a process for preparing a Δ9,11 steroid of the formula Ic,

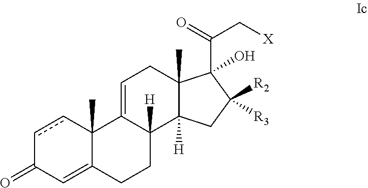

wherein:

The dotted line is a single or a double bond; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, substituted aryl, heteroaryl or C₁-C₈ alkoxy, or NR^{II}, wherein NR^{II} is a tertiary amine, wherein the tertiary amine is part of a linear or cyclic substructure.

Even more preferred is a process for preparing a Δ9,11 steroid of the formula Id,

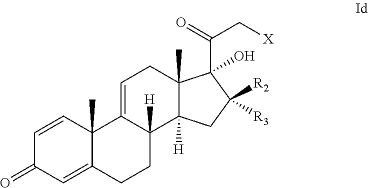

wherein:

One of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, subst. aryl, heteroaryl or C₁-C₆ alkoxy, or NR^{II}, wherein NR^{II} is a tertiary amine, wherein the tertiary amine is part of a linear or cyclic substructure.

Even more preferred is a process for preparing a Δ9,11 steroid of the formula Ie,

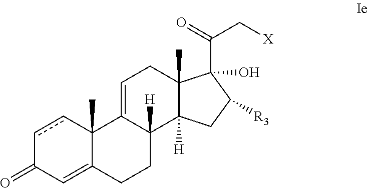

wherein:

The dotted line is a single or a double bond; R³ is CH₃ or H; and X is H, halo, OR, wherein R is H or C(O)R^{I}, and R^{I} is CF₃, C₁-C₈ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure.

Even more preferred is a process for preparing a Δ9,11 steroid of the formula If,

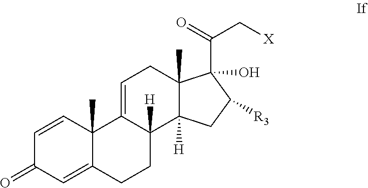

wherein:

R³ is CHs or H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure.

Particularly preferred is a process for preparing a Δ9,11 steroid of any of formulas Ia-If as defined above, wherein X is H, OH or OAc.

Most preferred is a process for preparing one of the Δ9,11 steroids:

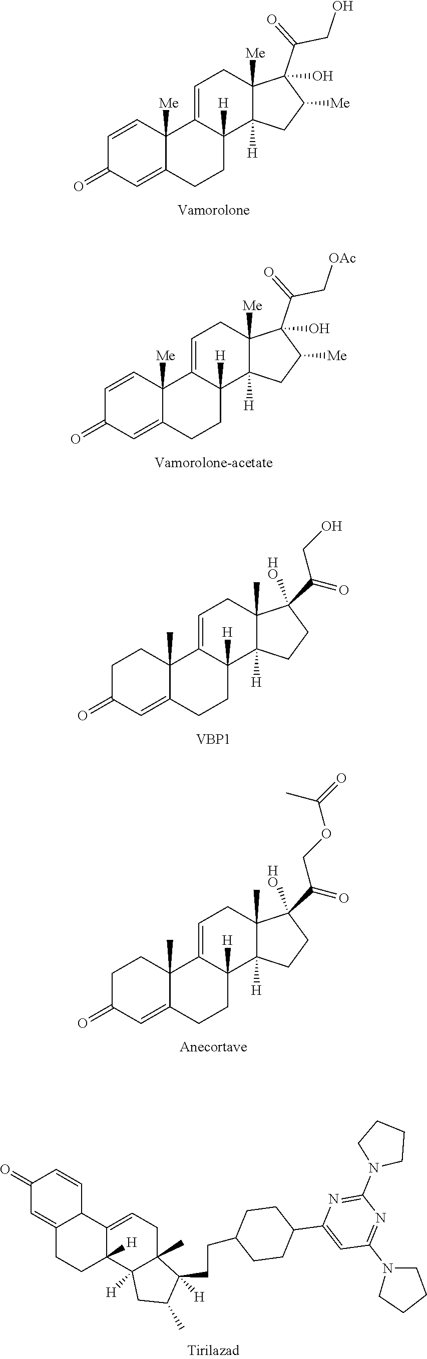

The instantly claimed process comprises treating an 9,11-epoxy-steroid of the formula II,

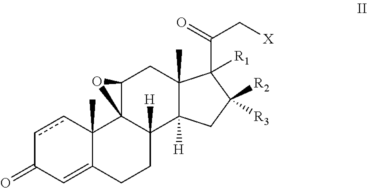

wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroarylor C₁-C₆ alkoxy, or NR^{II}, wherein NR^{II} is a tertiary amine, wherein the tertiary amine is part of a linear or cyclic substructure, with hydroiodic acid, HI, to form the compound of formula I.

Preferably, the instantly claimed process comprises treating an 9,11-epoxy-steroid of the formula IIa

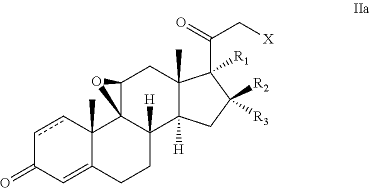

wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroarylor C₁-C₆ alkoxy, or NR^{II}, wherein NR^{II} is a tertiary amine, wherein the tertiary amine is part of a linear or cyclic substructure, with hydroiodic acid, HI, to form the compound of formula Ia.

More preferably, the instantly claimed process comprises treating an 9,11-epoxy-steroid of the formula IIb

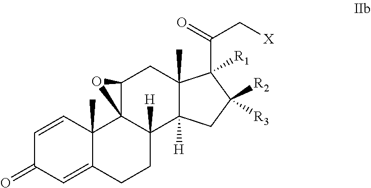

wherein R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure, with hydroiodic acid, HI, to form the compound of formula Ib.

Even more preferably, the instantly claimed process comprises treating an 9,11-epoxy-steroid of the formula IIc

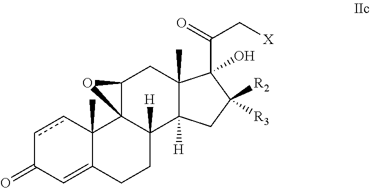

wherein the dotted line is a single or a double bond; one of R² or R³ is CH₃ and the other is H or both are H; and X is H, halo, OR, wherein R is H or C(O)R^{I}, and R^{I} is CF₃, C₁-C₆ alkyl, C₅-C₅-C₁₂ cycloalkyl, aryl, heteroarylor C₁-C₆ alkoxy, or NR^{II}, wherein NR^{II} is a tertiary amine, wherein the tertiary amine is part of a linear or cyclic substructure, with hydroiodic acid, HI, to form the compound of formula Ic.

Even more preferably, the instantly claimed process comprises treating an 9,11-epoxy-steroid of the formula IId

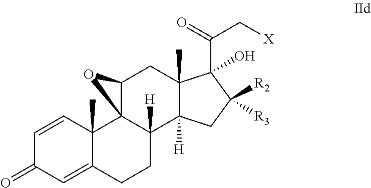

Wherein one of R² or R³ is CH₃ and the other is H or both are H; and X is H, halo, OR, wherein R is H or C(O)R^{I}, and R′ is CF₃, C₁-C₆ alkyl, C₅-C₁₂ cycloalkyl, aryl, heteroarylor C₁-C₆ alkoxy, or NR^{II}, wherein NR^{II} is a tertiary amine, wherein the tertiary amine is part of a linear or cyclic substructure, with hydroiodic acid, HI, to form the compound of formula Id.

Even more preferably, the instantly claimed process comprises treating an 9,11-epoxy-steroid of the formula IIe

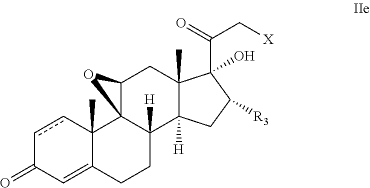

wherein the dotted line is a single or a double bond; R³ is CH₃ or H; and X is H, halo, OR, wherein R is H or C(O)R^{I}, and R′ is CF₃, C₁-C₆ alkyl, C₅-C₁₂ cycloalkyl, aryl, heteroarylor C₁-C₆ alkoxy, or NR^{II}, wherein NR^{II} is a tertiary amine, wherein the tertiary amine is part of a linear or cyclic substructure, with hydroiodic acid, HI, to form the compound of formula Ie.

Even more preferably, the instantly claimed process comprises treating an 9,11-epoxy-steroid of the formula IIf,

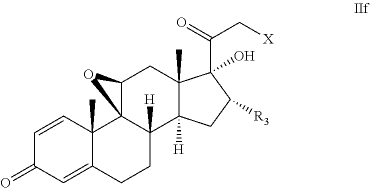

wherein R³ is CH₃ or H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure, with hydroiodic acid, HI, to form the compound of formula If.

More preferably, the instantly claimed process comprises treating a 9,11-epoxy-steroid of VBP1, Anecortave and Tirilazad, respectively, with with hydroiodic acid, HI, to form VBP1, Anecortave and Tirilazad, respectively.

Most preferably, the instantly claimed process comprises treating an 9,11-epoxy-steroid, which is 8-DM or 8-DM-acetate

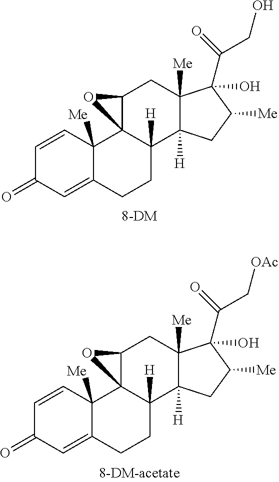

with hydroiodic acid, HI, to form Vamorolone and Vamorolone-acetate, respectively.

### DETAILS OF THE INVENTION

As used herein, the term “alkyl” means a straight or branched unsaturated hydrocarbon chain; preferred are C₁-C₆ alkyl having from 1 to 6 carbon atoms. The definition of alkyl and C₁-C₆-alkyl includes for example the meanings methyl, ethyl, n-, isopropyl, n-, iso-, sec- and t-butyl, n-pentyl, n-hexyl, 1,3-dimethylbutyl, and 3,3-dimethylbutyl.

The term “alkoxy” refers to an alkyl (carbon and hydrogen chain) group singularly bonded to oxygen, R—O; preferred are C₁-C₆ alkoxy groups having from 1 to 6 carbon atoms.

The term “halo” preferably means bromo, chloro or iodo.

“Cycloalkyl” groups in connection with the present invention are, unless defined otherwise, ring-shaped saturated hydrocarbon groups. Preferred are C₅-C₁₂ cycloalkyl having 5-12 ring C-atoms

“Aryl” in connection with the present invention is, unless defined otherwise, an aromatic hydrocarbon. Preferred is phenyl or substituted phenyl. “The term “heteroaryl” defines an aryl, which can have one, two or more heteroatoms which are selected from O, N, P and S and can optionally be substituted by further groups. Aryl may be substituted or unsubstituted. “Substituted aryl” means aryl as defined above but containing one or more substituents. Preferred are substituted aryl containing one or more alkoxy substituents, for instance methoxy groups, and/or one or more halogens, such as Cl. Heteroaryl may be substituted or unsubstituted. “Substituted heteroaryl” means heteroaryl as defined above but containing one or more substituents. Preferred are substituted heteroaryl containing one or more alkoxy substituents, for instance methoxy groups, and/or one or more halogens, such as Cl.

The term “tertiary amine” denotes an amino group in which the nitrogen atom is bonded to three organic radicals, where two of these radicals may also together be part of a ring. In the present invention, NR^{II} is a tertiary amine, wherein the tertiary amine is part of a linear or cyclic substructure. A preferred tertiary amine is a substituted or unsubstituted piperazin moiety, e.g.

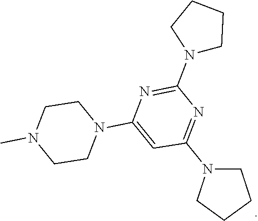 [SMILES: CN1CCN(c2cc(N3CCCC3)nc(N3CCCC3)n2)CC1]

The phrase “organic phase” is abbreviated in this specification by “OP”, “aqueous phase” by “AP”.

The term “pharmaceutical purity” defines an at least 99 wt % pure compound, e.g. Δ9,11 steroid, of the present invention, as determined by HPLC or other conventional methods. More preferably, the compound, e.g. Δ9,11 steroid, of the present invention, is at least 99.5 wt % pure as determined by HPLC or other conventional methods. Most preferably, the compound, e.g. Δ9,11 steroid, of the present invention, is at least 99.9 wt % pure as determined by HPLC or other conventional methods.

The following solvents and reagents employed in the process of the present invention are identified by the abbreviations indicated: ethyl acetate (EtOAc); acetic acid (HOAc); tetrahydrofuran (THF); dimethylsulfoxide (DMSO); triethylamine (Et₃N); diisopropylethylamine (Hünigs base); methanol (MeOH); 1,8-diazabicyclo [5.4.0]undec-7-ene (DBU); triphenylphosphine (PPh₃); diisopropyl ether (iPr₂O); dimethoxyethane (DME); t-butylmethyl ether (t-BuOMe); N,N-dimethylaminopyridine (DMAP); dimethylformamide (DMF); p-toluenesulfonyl chloride (TsCl); Bu₃Sn₂O (TBTO) Polymethylhydrosiloxane (PMHS).

The present invention comprises a process, designated Process A, deoxygenating the steroid of the formula II to form a Δ^{9,11} steroid of the formula I, as shown in Reaction Scheme A.

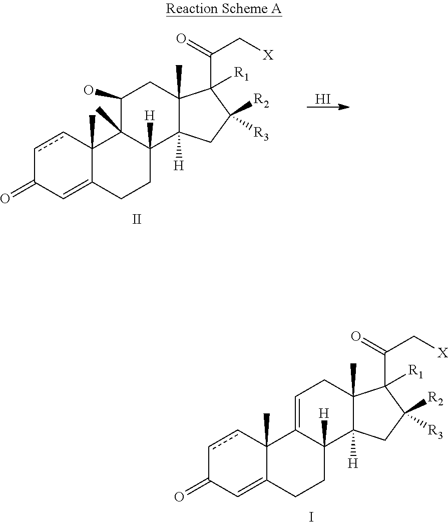

In Reaction Scheme A, a compound of formula II is treated with HI to form the compound of formula I. The present invention further comprises the process of Reaction Scheme A as shown above, wherein a compound of the formula IIa, II, IIc, IId or IIe is used instead of the compound of formula II.

As an example, Reaction Scheme A′ is shown. In Reaction Scheme A′, a compound of formula IId is treated with HI to form the compound of formula Id.

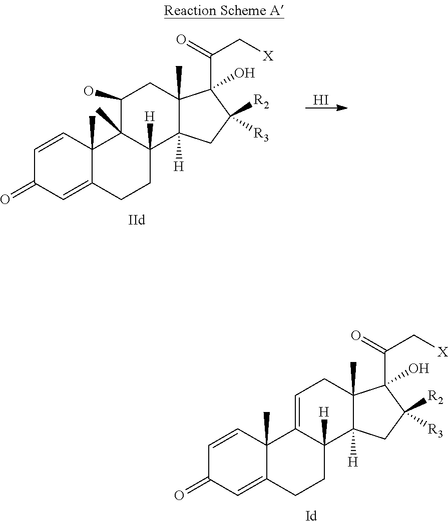

Most preferred is the synthesis of Vamorolone and Vamorolone Acetate (see Reaction Schemes B and B′), respectively.

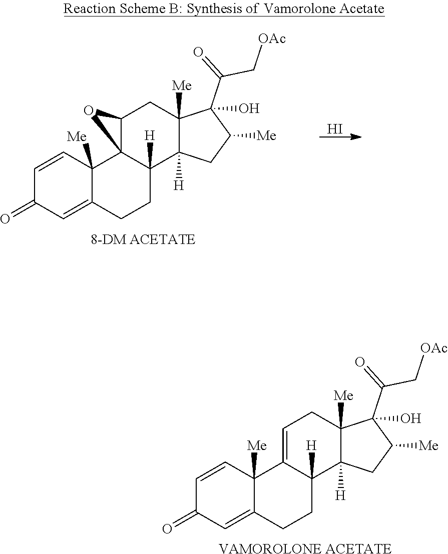

In Reaction Scheme B, 8-DM Acetate is treated with HI to form Vamorolone Acetate.

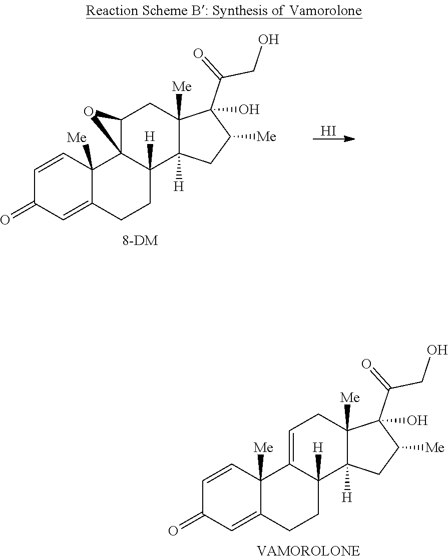

In Reaction Scheme B′, 8-DM is treated with HI to form Vamorolone. Surprisingly, the primary alcohol (21-hydroxy in 8-DM) does not undergo transformation to a corresponding alkyl iodide (21-I).

According to the present invention, the deoxygenation reactions of the present invention with HI can take place in any organic solvent. The solvent choice, however, is limited in as far as the solvent needs to be stable towards strong acid (HI) and the potentially formed 12, and should also not interfere with any of the reaction intermediates (e.g. iodohydrin). This reduces the number of solvent choices.

Any aromatic solvent, any chlorinated solvent (CH₂Cl₂, chloroform, 1,2-DCE etc.) and any nitrile solvent or mixtures of any of these solvents can be used. Preferred aromatic solvents are toluene, xylene, benzene, PhCF₃. Preferred chlorinated solvents are CH₂Cl₂, chloroform, 1,2-DCE. Preferred nitrile solvents are MeCN, propionitrile, butyronitrile

Three solvents are most preferred according to the present invention, i.e. toluene, MeCN and CH₂Cl₂. These solvents can be used alone or in mixtures of any of the three or a mixture of all tree solvents.

Alcoholic solvents, which are stable in the presence of HI, can be used as well. For example, CF₃CH₂OH and HFIP (hexafluoroisopropanol) may be used.

Etheral solvents, which are stable in the presence of HI, can be used as well. For example, DIPE (di-isopropylether) may be used.

The reaction can also be performed directly in an organic acid without any other solvent, e.g. directly in formic acid, acetic acid (i.e. AcOH), TFA etc. Examples of organic acids according to the present invention are acetic acid, TFA, formic acid, and propionic acid.

More preferably, the organic acid is AcOH. Organic acids, preferably AcOH, are good solvents for the reaction, as they facilitate elimination of the hydroxy-group at C11. However, in this embodiment, preferably an organic solvent (e.g. toluene, MeCN and CH₂Cl₂) needs to be added prior to quenching of the formed iodine with aqueous Na₂SO₃, to avoid by-product formation of insoluble residues.

Another solvent according to the present invention is H₂O, wherein the reaction works as well. Preferably, H₂O is used in a mixture with any of the organic acids as mentioned above. However, in H₂O without organic acids, I₂ will be formed and will precipitate from H₂O. This creates the need of removing precipitated I₂, quantitatively.

The reagent hydroiodic acid, HI, is preferably used in a high concentration aqueous form, more preferably in a concentration of 1%-70%, preferably 5%-70%, more preferably 10%-70%, even more preferably 30%-70% aqueous HI by mass, most preferably 48-57% aqueous HI or 64%-68% aqueous HI by mass.

The deoxygenation reaction with hydroiodic acid is performed at temperatures below room temperature RT (i.e. 25° C.), preferably below 15° C., more preferably below 10° C., most preferably below 5°, e.g. between 1 and 5° C.

To purify the compound of formula II, the final compound is recrystallized. Recrystallization may be performed from polar solvents such as water or alcohols and mixtures thereof and alternatively using Acetonitrile, Acetone, Methyl ethyl ketone (MEK), Methyl isopropyl ketone (MIK) and mixtures thereof. Preferably, re-crystallization is performed from isopropanol (iPr-OH) or a mixture of iPr-OH and water. More preferably recrystallization takes place in a mixture of iPr-OH: water between 60:40 wt % and 100:0 wt %, preferably between 80:20 wt % and 100:0 wt %

Typically the last crystallization of crude Vamorolone from isopropanol or isopropanol water mixtures is able to provide a purity upgrade of more than 1 wt %, e.g. from 98.5 wt % to between 99.6 wt % and 99.9 wt % while still providing good yields above 90%. This final polishing step by crystallization allows excellent control of the impurity profile of the final API.

In a further aspect of the present invention a protecting group for the primary hydroxy group on C-21 can be added in place of moiety X before the deoxygenation of formula II, IIa, IIb, IIc, IId, IIe or IIf, respectively.

The protecting group in place of X, is OR, wherein R is C(O)R^{I}, wherein R^{I} is CF₃, C₁-C₆ alkyl, C₅-C₁₂ cycloalkyl, aryl, heteroaryl or C₁-C₆ alkoxy.

After the addition of the protecting group, the deoxygenation according to the present invention is performed, followed by a de-protection step, i.e. a step in which the protecting group is removed.

In this step moiety X, which is OR, wherein R is C(O)R^{I}, and R^{I} is CF₃, C₁-C₆ alkyl, C₅-C₁₂ cycloalkyl, aryl, heteroaryl or C₁-C₆ alkoxy, is converted into X, which is OH. OH can furthermore be protected using methoxymethyl ether, tetrahydropyranyl ether, t-butyl ether, benzyl ether, dimethoxybenzyl ether, t-butyldimethylsilyl ether, t-butyldiphenylsilyl ether, acetic acid ester, pivalic acid ester, benzoic acid ester, acetonide or benzylidene acetal or any other conventional protecting groups.

In a preferred embodiment, 8-DM is acetylated before the deoxygenation with HI. The obtained 8-DM Acetate is deoxygenated with HI to Vamorolone Acetate and subsequently de-acetylated to form Vamorolone. If Vamorolone Acetate is partially deacetylated during the HI reaction, it is optional to re-acetylate the Vamorolone partially de-acetylated, again. Quantitative acetylation may be intended to quantitatively isolate Vamorolone Acetate and obtain a quantitative total synthesis of Vamorolone, if the synthesis involves Vamorolone Acetate formation by adding HI to 8-DM acetate.

The acetylation procedure can be performed using known reaction conditions specified in this patent in the experimental section or conditions known to the skilled person. However, particularly preferred is a reaction using Ac₂O in an organic solvent, preferably in Acetonitrile. Most preferably, AC₂O in Acetonitrile is used with catalytic amounts of DMAP. The 8-DM-Acetate may be obtained as a crystalline product after aqueous quenching. The acetylation may be performed at room temperature. Preferred temperature ranges are between 20 and 30° C., more preferably 22-25° C.

The de-acetylation hydrolysis procedure can be performed using reactions known in the art. However, particularly preferred is de-acetylation with K₂CO₃ in an alcohol or an alcohol/water-mixture. Most preferred is K₂CO₃ in MeOH/H₂O. In a further embodiment, Vamorolone Acetate is de-acetylated with KOH in an alcohol or an alcohol/water-mixture. Most preferred is KOH in MeOH.

The temperature of the de-acetylation reaction is lower than RT, preferably lower than 15° C., more preferably lower than 10° C., most preferably between 0°-5° C.

In a preferred embodiment of the present invention, the Δ9,11 steroid is prepared by deoxygenation of a respective 9,11-epoxy steroid using HI, wherein the Δ9,11 steroid is prepared using aqueous HI at a concentration of between 48 and 68 wt %, in MeCN, CH₂Cl₂, or Toluene; or a mixture of two or all three thereof, in the presence or absence of an organic acid, wherein treatment with HI is carried out at a temperature of below 15° C.

In a more preferred embodiment of the present invention, the Δ9,11 steroid is prepared by deoxygenation of a respective 9,11-epoxy steroid using HI, wherein the Δ9,11 steroid is prepared using aqueous HI at a concentration of between 48 and 68 wt %, in MeCN, CH₂Cl₂, or Toluene; or a mixture of two or all three thereof, in the presence or absence of an organic acid, which is acetic acid, wherein treatment with HI is carried out at a temperature of below 15° C.

In an even more preferred embodiment of the present invention, the Δ9,11 steroid is prepared by deoxygenation of a respective 9,11-epoxy steroid using HI, wherein the Δ9,11 steroid is prepared using aqueous HI at a concentration of between 48 and 68 wt %, inToluene; in the presence or absence of an organic acid, which is acetic acid, wherein treatment with HI is carried out at a temperature of below 15° C.

In an even more preferred embodiment of the present invention, the Δ9,11 steroid is prepared by deoxygenation of a respective 9,11-epoxy steroid using HI, wherein the Δ9,11 steroid is prepared using aqueous HI at a concentration of between 48 and 68 wt %, in toluene; in the presence or absence of an organic acid, which is acetic acid, wherein treatment with HI is carried out at a temperature of below 15° C., wherein if X is OH, the 9,11 epoxy steroid is acetylated before the treating of the 9,11 epoxy steroid with HI. In this embodiment, the Δ9,11 steroid obtained after treating the 9,11 epoxy steroid with HI, is preferably de-acetylated.

Most preferably, the processes of the present invention produce Vamorolone or Vamorolone Acetate, more preferably from the commercial 8-DM.

The Δ9,11 steroids obtained by the processes of the present invention are ideally pharmaceutically pure Δ9,11 steroids. Purification can be performed by recrystallization of any of the Δ9,11 steroids obtained by any of the processes of the present invention, which are typically obtained as crude Δ9,11 steroids, in iPrOH or a mixture of iPrOH and water.

### EXAMPLES

### Example 1: Unsuccessful Attempts

### 1.1 Unsuccessful Conditions

Deoxygenation was tried but found to be unsuccessful with the following reactions:
a) Rhenium-catalysts: CH₃ReO₃, Re₂O₇ or perrhenic acid) in combination with Triphenylphosphite P(OPh) 3 in toluene at reflux,
b) Electrochemical Reduction with aq. NH₄Br/THF with Zn-electrodes. Constant Current, 30 mA, and
c) PPh₃ & I₂ in MeCN.

None of these reactions known from the literature turned out to be successful.

1.2 Useful but Complex and Toxic Reaction with HBr

Alternative deoxygenation conditions using HBr followed by tin mediated PMHS reduction and subsequent water elimination involves large quantities of a tin reagent and liquid sulfur dioxide as the solvent for the elimination step. Neither the use of tin nor toxic gases as solvents or reagents is desirable for the downstream manufacturing of an API if it can be avoided.

Thus there is a need for a new process to circumvent the use of hazardous and toxic chemicals towards the end of the chemical sequence to Vamorolone.

Furthermore, the HBr route to Vamorolone has the potential to form PMIs (Potentially Mutagenic Impurities) such as allylic bromides of Vamorolone that could represent a patient risk. These impurities are hard to trace and must be controlled rigorously to very low contamination levels.

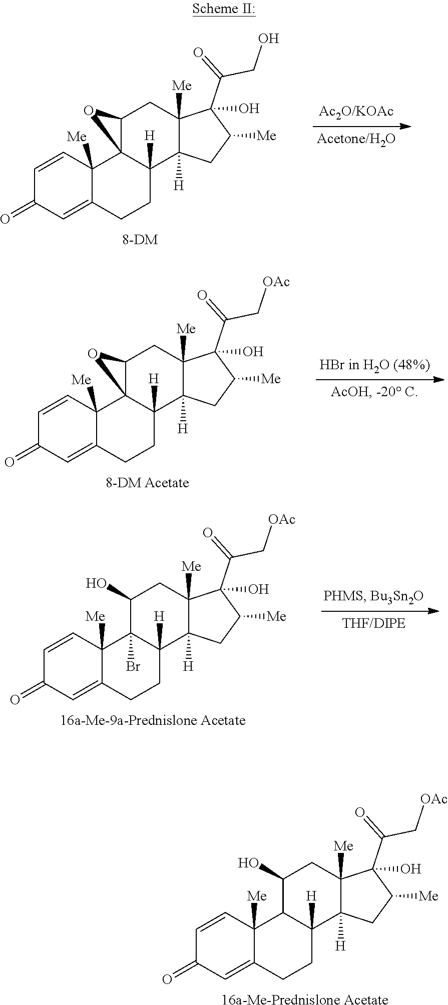

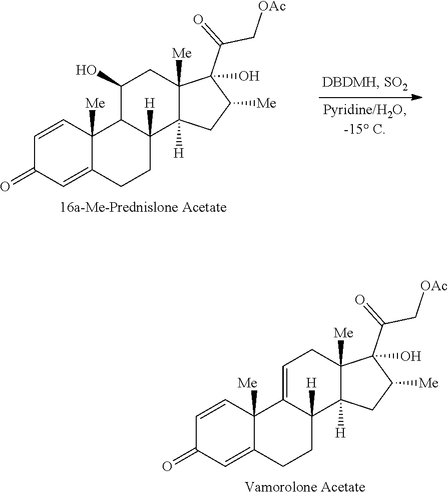

The steps of scheme 2 turned out to be possible, but unintended for several reasons: Debromination with Bu₂Sn₂O comprises the in situ generation of Bu₃SnH, which is toxic. Bu₃Sn₂O (TBTO) used as biocid in ship paintings to reduce biofouling. TBT is highly toxic itself towards nontarget organisms. Toxic effects occur already at 1 nano-gram per liter of water. SO₂ is toxic and it represents a safety hazard in a manufacturing plant and needs specific safety installations, controls and waste treatment facilities.

1.3 Useful but Imperfect Reaction with “TMSI”

Deoxygenation was tried, but found to be uncomfortable, by use of NaI (3.0 eq.) & TMSCl (1.5 eq.) in MeCN. After in situ formation of TMS-iodohydrin, and subsequent elimination the deoxygenation took place:

However, incomplete transformation of 8-DM-Acetate and a non-satisfactory resulting impurity profile required a new solution. Thus, the inventors sought to provide a new, even simpler and more straight forward and scalable synthesis.

### Example 2: Synthesis of the Present Invention

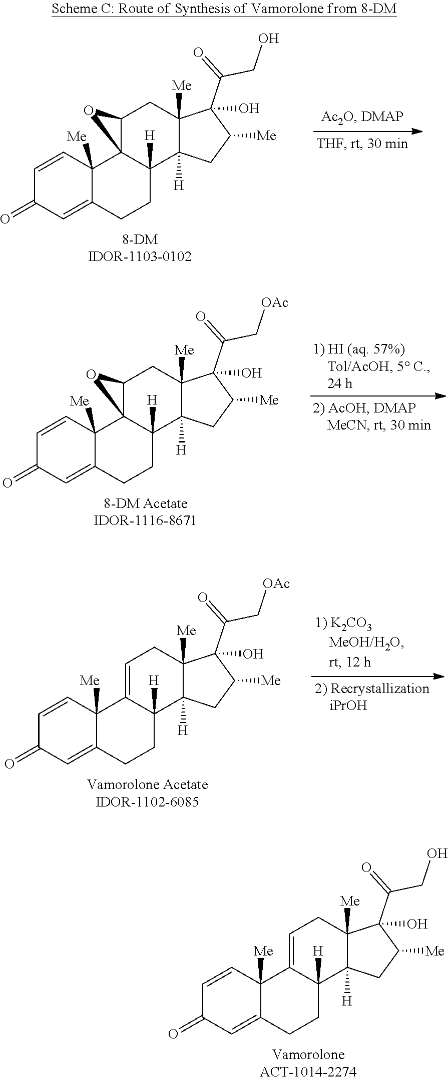

Vamorolone was synthesized in three synthetic steps from commercially available 8-DM.

The synthetic route started with the acetylation of 8-DM using acetic anhydride and catalytic DMAP in THF, followed by crystallization of 8-DM Acetate after aqueous quench. Then, a deoxygenation reaction converted 8-DM Acetate directly into Vamorolone Acetate. This deoxygenation proceeded via initial formation of an iodohydrin with excess aq. HI, followed by simultaneous I₂ and H₂O-elimination to give Vamorolone Acetate. During the reaction, partial de-acetylation occurred (20-25%) and therefore re-acetylation with acetic anhydride was necessary. After completed re-acetylation, Vamorolone Acetate was directly crystallized by addition of H₂O. Finally, the acetate group is cleaved under basic conditions to give crude Vamorolone, which was recrystallized from iPrOH to obtain the pure product.

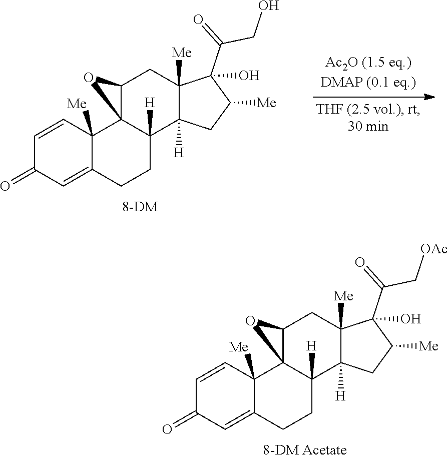

A 10 L glass dj (double jacketed reactor)-reactor was charged with 8-DM (490 g, 1.32 mol, 1.0 eq.) and DMAP (16.1 g, 0.132 mmol, 0.10 eq.). THF (1.25 L, 2.5 vol.) was added at IT=20-25° C. Then, Ac₂O (201 g, 187 mL, 1.97 mol, 1.5 eq.) was added dropwise over 20-40 min, keeping IT below 30° C. during the addition. After complete addition, the reaction mixture was stirred at IT=20-25° C. for 30 min. IPC control by LC/MS indicated >99% conversion of 8-DM to 8-DM Acetate.

The reaction mixture was quenched by dropwise addition of H₂O (4.9 L, 10 vol.) over 30-45 min, keeping IT below 25° C. The resulting aqueous suspension was aged at IT=20-25° C. for 1 h. The product was filtered off, washed with H₂O (3×0.5 L), and dried on a rotary evaporator (900-10 mbar, 65° C. bath temperature) to provide 8-DM Acetate (539 g, 1.30 mol, 99% yield, >99% a/a, 98% w/w) as a white solid (cryst 1 #1).

### Analytical Data:

LC/MS column: Zorbax RRHD SB-Aq, 2.1×50 mm, 1.8 μm
Program: G_005% B_TFA_0,800 ml_2.00 min
Eluent A: Water/TFA 100:0.04, Eluent B: Acetonitrile
IPC preparation for LC/MS
10 microliter in 1 mL H2O:MeCN 1:1
Conversion was determined with respect to consumption of 8-DM relative to formation of 8-DM Acetate.
Detected mass: [M+1]=373.19 for 8-DM and [M+1]=415, 19 8-DM Acetate.

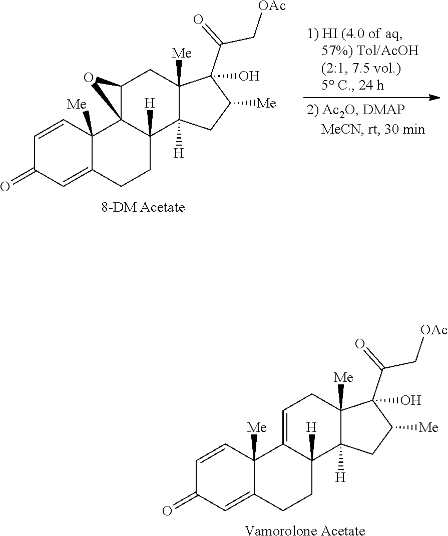

A 10 L glass dj-reactor was charged with 8-DM Acetate (500 g, 1.21 mol, 1.0 eq.). Toluene (2.5 L, 5 vol.) was added. The suspension was cooled to IT=0-5° C. and then a solution of 57% aqueous HI (1.08 kg, 637 mL, 4.83 mol, 4.0 eq.) in AcOH (1.25 L, 2.5 vol.) was added via peristaltic pump over 45-60 min, keeping IT below 5° C. during the addition. The resulting dark purple to brown solution was stirred at IT=3-5° C. for 24 h. IPC control by LC/MS indicated >98% conversion of 8-DM Acetate/intermediate iodohydrin to Vamorolone Acetate/Vamorolone.

The reaction mixture was quenched by dropwise addition of 25% aq. Na₂SO₃ solution (2.0 L, 4 vol.) over 10-20 min, keeping IT below 15° C. After complete addition, EtOAc (1.0 L, 2 vol.) was added and the biphasic mixture was warmed to IT=15-20° C. Stirring was stopped and the phases were separated (Organic Phase 1 and aqueous Phase 1; goal pH of the aqueous Phase 1:2; aqueous Phase 1 disposed). 25% aq. Na₂SO₃ solution (1.25 L, 2.5 vol.) was added to Organic Phase 1 and the biphasic mixture was stirred at IT=15-20° C. for 5 min, stirring was stopped and phases separated (Organic Phase 1 and aqueous Phase 2; goal pH aqueous Phase 2:4-5; aqueous Phase 2 disposed). 25% aq. Na₂SO₃ solution (1.25 L, 2.5 vol.) was added to Organic Phase 1 and the biphasic mixture was stirred at IT=15-20° C. for 5 min, stirring was stopped and phases separated (Organic Phase 1 and aqueous Phase 3; goal pH aqueous Phase 3:5-6; aqueous Phase 3 disposed). H₂O (0.5 L, 1.0 vol.) was added to Organic Phase 1 and the biphasic mixture was stirred at IT=15-20° C. for 5 min, stirring was stopped and phases separated (Organic Phase 1 and aqueous Phase 4; goal pH aqueous Phase 4:5-6; aqueous Phase 4 disposed).

A slight vacuum was applied to the double-jacketed reactor (100-150 mbar), containing Organic Phase 1, and toluene was distilled off at 70° C. jacket temperature (ET) from the reaction mixture with continuous addition of MeCN, and the distillation continued until target residual toluene value has been reached (goal: less than 5% toluene according to 1H-NMR of reaction mixture. Final volume in reactor after distillation: ca. 3.5 L (7.5 vol.).

Once toluene was removed, the vacuum was broken with N₂ and resulting fine suspension cooled to IT=20-25° C. At this point, the amount of Vamorolone was assessed by IPC (typical ratio: Vamorolone Acetate to Vamorolone:75:25; x=25% a/a). DMAP (3.7 g, 0.0302 mol, 0.025 eq.) was added, followed by slow addition of Ac₂O (61.6 g, 57 mL, 0.603 mol, 0.5 eq.) over 5-10 min at IT=20-25° C. After complete addition of Ac₂O, the reaction mixture was stirred for 30 min at IT=20-25° C. IPC control by LC/MS indicated ≤2% a/a Vamorolone (ratio: Vamorolone Acetate to Vamorolone:98.5:1.5).

The reaction mixture was quenched by slow addition of H₂O (4.9 L, 10 vol.) over 15-30 min, keeping IT below 25° C. The resulting aqueous suspension was cooled to IT=0-5° C. and aged at this temperature for 2 h. The product was filtered off, washed with H₂O/MeCN 4:1 (2×0.5 L), and dried on a rotary evaporator (900-10 mbar, 65° C. bath temperature) to provide Vamorolone Acetate (301 g, 0.76 mol, 63% yield, 98% a/a, 98% w/w) as an off-white solid (cryst 1 #1).

Over the course of the reaction, partial de-acetylation of Vamorolone Acetate to Vamorolone was observed (between 20-25% a/a). Therefore, after aq. workup and solvent switch to MeCN, the ratio of Vamorolone Acetate to Vamorolone was assessed by LC/MS (in % a/a), and the following amounts of DMAP and Ac2O were added:
x=amount of Vamorolone in % a/a (e.g. x=20% a/a)
DMAP eq.=(0.1·x)/100 (e.g. 0.02 eq.)
Ac2O eq.=(2.0·x)/100 (e.g. 0.40 eq.)

### Analytical Data

LC/MS column: Zorbax RRHD SB-Aq, 2.1×50 mm, 1.8 μm
Program: G_005% B_TFA_0,800 ml_2.00 min
Eluent A: Water/TFA 100:0.04, Eluent B: Acetonitrile
IPC preparation for LC/MS
10 microliter in 1 mL H₂O:MeCN 1:1
Conversion was determined with respect to consumption of the sum of (8-DM Acetate+intermediate iodohydrin) relative to the sum of (Vamorolone Acetate+Vamorolone).
Detected mass: [M+1]=415.19 for 8-DM Acetate, [M+1]=357.28 for Vamorolone, 399.20 for Vamorolone Acetate and 543.12 for intermediate lodohydrin

### 2.3 De-Acetylation

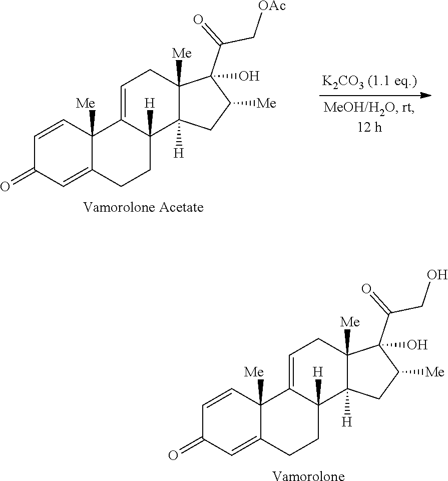

A 10 L glass dj-reactor was charged with Vamorolone Acetate (280 g, 0.703 mol, 1.0 eq.). MeOH (1.54 L, 5.5 vol.) was added. The suspension was cooled to IT=0-5° C. and then a solution of K₂CO₃ (107 g, 0.773 mol, 1.1 eq.) in H₂O (0.7 L, 2.5 vol.) was added dropwise via peristaltic pump over 20-40 min, keeping IT below 10° C. during the addition. After complete addition, the reaction mixture was warmed IT=20-25° C. and stirred for 5 h. IPC control by LC/MS indicated 99.3% conversion of Vamorolone Acetate to Vamorolone.

The reaction mixture was cooled to IT=15-17° C. and quenched by dropwise addition of 1 M aq. HCl (950 mL, 0.95 mol, 1.35 eq.) over 20-40 min, keeping IT below 20° C. during the addition (goal pH: 5-6). The resulting aqueous suspension was aged at IT=15-20° C. for 12 h. The product was filtered off, washed with H₂O/MeOH 2:1 (3×0.3 L), and dried on a rotary evaporator (900-10 mbar, 65° C. bath temperature) to provide Vamorolone (241.5 g, 0.68 mol, 96% yield, >99% a/a, 98% w/w) as a slightly yellow solid (crude 1 #1).

### Analytical Data

LC/MS column: Zorbax RRHD SB-Aq, 2.1×50 mm, 1.8 μm
Program: G_005% B_TFA_0,800 ml_2.00 min
Eluent A: Water/TFA 100:0.04, Eluent B: Acetonitrile
IPC preparation for LC/MS
10 microliter in 1 mL H₂O:MeCN 1:1
Conversion was determined with respect to consumption of Vamorolone Acetate relative to formation of Vamorolone.

### 2.4 Recrystallization

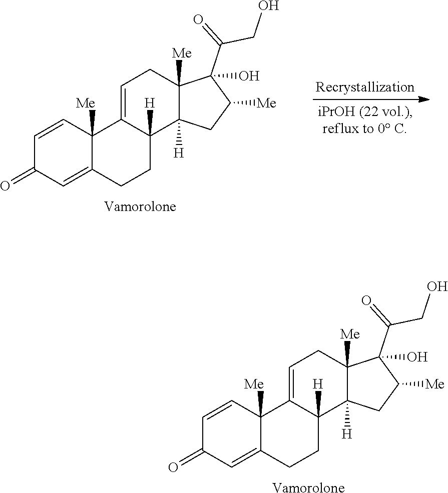

A 10 L glass dj-reactor was charged with Vamorolone (230 g, 0.645 mol, 1.0 eq.). iPrOH (5 L, 22 vol.) was added. The suspension was heated to reflux (jacket temperature ET=97° C.) and stirred until complete dissolution of Vamorolone occurred (10-15 min on this scale). After complete dissolution, the clear yellow solution was slowly cooled to IT=0-5° C. over the course of 12 h and then aged at IT=0-5° C. for 1 h. The recrystallized product was filtered off, washed with cold iPrOH (2×250 mL), and dried on a rotary evaporator (900-10 mbar, 65° C. bath temperature) to provide Vamorolone (201 g, 87% recovery, >99% a/a, 99% w/w) as an off white glimmery solid (cryst 1 #1).

Iso-propanol (iPrOH) was found to the best solvent for recrystallization with excellent purity upgrading properties (by rejection of impurities), although a high dilution is necessary to completely dissolve the crude Vamorolone at reflux temperature. Higher concentrations for the recrystallization satisfactory results are obtainable using mixtures of isopropanol and water. Maximum solubility of Vamorolone was determined to be at reflux of a 80:20 (isopropanol:water) mixture.

### 2.5 Improving Purity by Charcoal Treatment

To improve Vamorolone Acetate purity, a charcoal treatment was envisaged. Two options were found to be useful.

### Option 1: Charcoal Treatment on Isolated Vamorolone Acetate

Vamorolone Acetate (10 g) from HI step (ELN293-1469.1) was suspended in MeCN (100 mL, 10 vol.) and H₂O (10 mL, 1 vol.). The suspension was heated to IT=60-65° C. and stirred until Vamorolone Acetate was completely dissolved. Then, charcoal (1.0 g, 10% w/w) was added and stirred for 1 h at IT=60-65° C. The mixture was filtered hot through a Whatman glass microfiber filter into a 500 mL round-bottom flask. Additional H₂O (90 mL, 9 vol.) was slowly added to induce crystallization of Vamorolone Acetate. The suspension was slowly cooled to 0° C. and aged for 1 h. The white solid was filtered off, washed with additional MeCN:H2O 1:4 (2×10 mL) and dried under reduced pressure on the rotavap (65° C.) for extended amount of time (9.0 g, 90% recovery).

### Option 2: Charcoal Treatment During HI Step

A 1 L glass dj-reactor was charged with 8-DM Acetate (30.0 g, 72.4 mmol, 1.0 eq.). Toluene (150 mL, 5 vol.) was added. The suspension was cooled to IT=0-5° C. and then a solution of 57% aqueous HI (65.0 g, 38.2 mL, 290 mmol, 4.0 eq.) in AcOH (75 mL, 2.5 vol.) was added via peristaltic pump over 45-60 min, keeping IT below 5° C. during the addition. The resulting dark purple to brown solution was stirred at IT=3-5° C. for 24 h. IPC control by LC/MS indicated >98% conversion of 8-DM Acetate/intermediate iodohydrin to Vamorolone Acetate/Vamorolone.

The reaction mixture was quenched by dropwise addition of 25% aq. Na₂SO₃ solution (120 mL, 4 vol.) over 10-20 min, keeping IT below 15° C. After complete addition, EtOAc (60 mL, 2 vol.) was added and the biphasic mixture was warmed to IT=15-20° C. Stirring was stopped and the phases were separated (Organic Phase 1, i.e. OP1 and aqueous Phase 1, i.e. AP1; goal pH AP1:2; AP1 disposed). 25% aq. Na₂SO₃ solution (75 mL, 2.5 vol.) was added to OP1 and the biphasic mixture was stirred at IT=15-20° C. for 5 min, stirring was stopped and phases separated (OP1 and AP2; goal pH AP2:4-5; AP2 disposed). 25% aq. Na2SO3 solution (75 mL, 2.5 vol.) was added to OP1 and the biphasic mixture was stirred at IT=15-20° C. for 5 min, stirring was stopped and phases separated (OP1 and AP3; goal pH AP3:5-6; AP3 disposed). H2O (30 mL, 1.0 vol.) was added to OP1 and the biphasic mixture was stirred at IT=15-20° C. for 5 min, stirring was stopped and phases separated (OP1 and AP4; goal pH AP4:5-6; AP4 disposed).

A slight vacuum was applied to the double-jacketed reactor (100-150 mbar), containing OP1, and toluene was distilled off at 70° C. jacket temperature (ET) from the reaction mixture with continuous addition of MeCN, and the distillation continued until target residual toluene value has been reached (goal: less than 5% toluene according to 1H-NMR of reaction mixture. Final volume in reactor after distillation: ca. 225 mL (7.5 vol.))

Once toluene was removed, the vacuum was broken with N₂ and resulting fine suspension cooled to IT=20-25° C. At this point, the amount of Vamorolone was assessed by IPC (ratio: Vamorolone Acetate to Vamorolone:75:25; x=25% a/a). DMAP (221 mg, 1.81 mmol, 0.025 eq.) was added, followed by slow addition of Ac₂O (3.7 g, 3.4 mL, 36.2 mmol, 0.5 eq.) over 2-3 min at IT=20-25° C. After complete addition of Ac₂O, the reaction mixture was stirred for 30 min at IT=20-25° C. IPC control by LC/MS indicated less than 1% a/a Vamorolone.

The reaction mixture was quenched by slow addition of H₂O (30 mL, 1 vol.) over 5-10 min. The suspension was heated to IT=60-65° C. and stirred until complete dissolution has occurred. Then, charcoal (3.0 g, 10% w/w) was added and stirred for 1 h at IT=60-65° C. The mixture was filtered hot through a Whatman glass microfiber filter into a 1 L round-bottom flask. Additional H₂O (195 mL, 6.5 vol.) was slowly added to induce crystallization of Vamorolone Acetate. The suspension was slowly cooled to 0° C. and aged for 1 h. The white solid was filtered off, washed with additional MeCN:H₂O 1:4 (2×40 mL) and dried under reduced pressure at the rotavap (65° C.) for extended amount of time (18.7 g, 46.9 mmol, 65% yield, >99% a/a).

1. A method of making a Δ9,11 steroid from a 9,11 epoxy steroid, comprising treating the 9,11 epoxy steroid with hydroiodic acid (HI).

2. The method of claim 1, wherein the Δ9,11 steroid is a steroid of formula I, 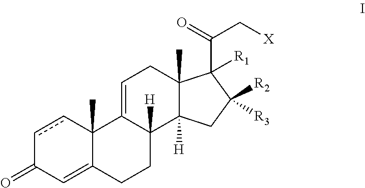
wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl or C₁-C₆ alkoxy, C₅-C₁₂ cycloalkyl, aryl, heteroaryl or NR^{II}, wherein NR^{II} is a tertiary amine, wherein the tertiary amine is part of a linear or cyclic substructure.

3. The method of claim 1, wherein the Δ9,11 steroid is a steroid of formula If, 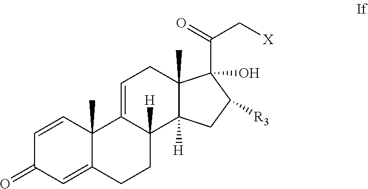
wherein R³ is CH₃ or H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure.

4. The method of claim 3, wherein the Δ9,11 steroid is Vamorolone or Vamorolone Acetate,

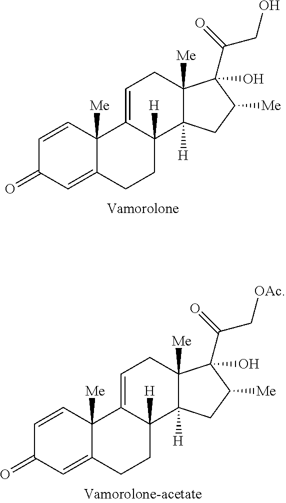

5. A process for preparing a Δ9,11 steroids of formula I, 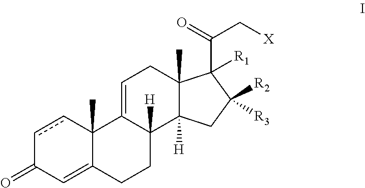
wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure,
the process comprising treating an 9,11 epoxy steroid of formula II 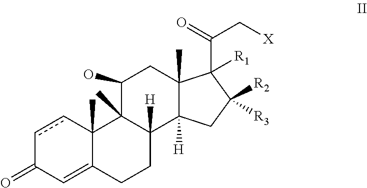
wherein the dotted line is a single or a double bond; R¹ is H or OH; one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine,
with hydroiodic acid, HI, to form the Δ9,11 steroid of formula I.

6. The process of claim 5, wherein the Δ9,11 steroid is a compound of formula Id, 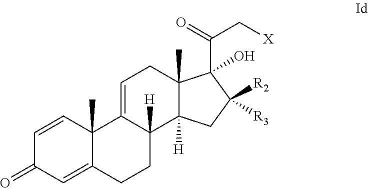
wherein one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure, and
wherein the 9,11 epoxy steroid is a compound of formula IId, 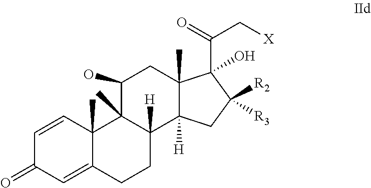
wherein one of R² or R³ is CH₃ and the other is H or both are H; and X is H, halo, OR, wherein R is H or C(O)R^{I}, and R^{I} is CF₃, C₁-C₆ alkyl, C₅-C₁₂ cycloalkyl, aryl, heteroaryl or C₁-C₆ alkoxy, or NR^{II}, wherein NR^{II} is a tertiary amine, wherein the tertiary amine is part of a linear or cyclic substructure, with hydroiodic acid, HI, to form the compound of formula Id.

7. The process of claim 6, wherein the Δ9,11 steroid is a compound selected from Vamorolone or Vamorolone Acetate 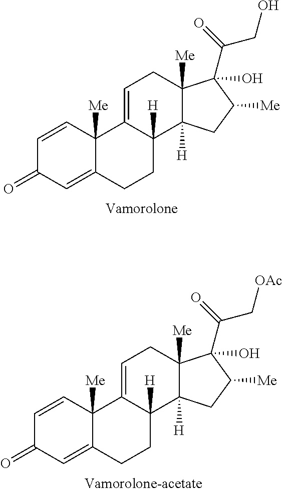
and the 9,11 epoxy steroid is a compound of 8-DM and 8-DM acetate, respectively.

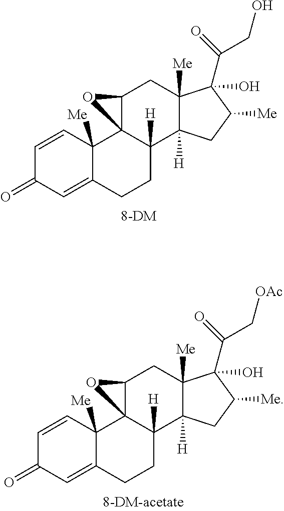

8. The process of claim 5, wherein the Δ9,11 steroid is prepared using aqueous HI in an organic solvent and in the presence of an organic acid or without the addition of an organic acid and wherein treatment is carried out at a temperature of below 15° C.

9. The process of claim 8, wherein the organic solvent is MeCN, CH₂Cl₂, or Toluene; or a mixture of two or all three thereof, and
wherein the organic acid is acetic acid.

10. The process of claim 5, wherein the Δ9,11 steroid is prepared using aqueous HI, at a concentration of between 48 and 68 wt %, in toluene.

11. The process of claim 5, wherein if X is OH, the 9,11 epoxy steroid is acetylated before the treating of the 9,11 epoxy steroid with HI.

12. The process of claim 11, wherein the Δ9,11 steroid obtained after treating the 9,11 epoxy steroid with HI, is de-acetylated.

13. The process of claim 5, further comprising a step of recrystallizing the Δ9,11 steroid obtained, in iPrOH or a mixture of iPrOH and water, thereby preparing a pharmaceutically pure Δ9,11 steroid.

14. A process of purifying Vamorolone comprising a step of re-crystallizing crude Vamorolone in isopropanol or in a mixture of water and isopropanol, thereby forming purified Vamorolone of 99.5 wt % purity or more.

15. A Δ9,11 steroid produced by the process of claim 5.

16. A pharmaceutically pure Δ9,11 steroid produced by the process of claim 13.

17. A pharmaceutical composition comprising the compound of claim 15.

18. A Δ9,11 steroid produced by the method of claim 1.

19. a purified Vamorolone produced by the process of claim 14.

20. A pharmaceutical composition comprising the compound of claim 16.
